(19) United States
(12) Patent Application Publication
(10) Pub. No.: US 2013/0131491 A1
(43) Pub. Date: May 23, 2013

(54) Tumor Response Prediction to Therapy

(71) Applicant: The Board of Trustees of the Leland Stanford Junio, Palo Alto, CA (US)

(72) Inventors: Phuoc Tho Tran, Ellicott City, MD (US); Pavan Bendapudi, Cambridge, MA (US); Hen-Tzu Lin, San Jose, CA (US); David S. Paik, Half Moon Bay, CA (US); Dean W. Felsher, San Mateo, CA (US)

(21) Appl. No.: 13/680,090

(22) Filed: Nov. 18, 2012

Related U.S. Application Data
(60) Provisional application No. 61/629,428, filed on Nov. 18, 2011.

(51) Int. Cl. A61B 5/00; A61B 6/03; A61B 5/055

(52) U.S. Cl. CPC A61B 5/72; A61B 5/055; A61B 6/037
USPC 600/410; 600/407; 600/425

(57) ABSTRACT

Tumor responses to a therapy can be predicted in a more objective and quantitative fashion allowing doctors to make earlier determinations of how well a tumor is responding to therapy. If a patient were not responding well, valuable time could be saved and the patient could be switched to a more efficacious therapy. Tumor response predictions to therapy are determined from a combination of (i) the tumor volumes over time, (ii) the cellular proliferation over time and (iii) the cellular apoptosis over time.

### CROSS-REFERENCE TO RELATED APPLICATIONS

This application claims priority from U.S. Provisional Patent Application 61/629,428 filed Nov. 18, 2011, which is incorporated herein by reference.

### STATEMENT OF GOVERNMENT SPONSORED SUPPORT

This invention was made with Government support under contract CA114747 awarded by National Institutes of Health (NIH). The Government has certain rights in this invention.

### FIELD OF THE INVENTION

This invention relates to cancer therapy. In particular, the invention relates to methods and systems to predict tumor response to therapy.

### BACKGROUND OF THE INVENTION

In general, technology that could predict the efficacy of targeted therapeutics would be highly useful in the development of new therapies and evaluation of therapies. In addition, because targeted therapies are very expensive and are usually only effective to treat a very specific subpopulation of cancer patients, it is important to develop strategies to rapidly discriminate when these agents are effective to help a particular patient.

Some cancer cells are dependent or “addicted” to the continued activity of oncoproteins. Drugs that target these oncoproteins induce the addicted cancer cells to die rapidly, which is referred to as “oncogene addiction”. Marked clinical responses have been reported in some cancer patients, particularly those with lung cancer, after treatment with drugs targeting oncoproteins. However, only a distinct subset of human cancer patients have tumors that exhibit this behavior of oncogene addiction. Therefore, the ability to predict when a tumor will exhibit oncogene addiction would be useful not only for developing new oncoprotein-targeted therapies, but also for selecting which cancer patients are likely to respond best to such drugs.

The ideal predictive method would be noninvasive, generate reproducible measurements, and be widely available using technology and clinical skills generally available to most hospitals. Previous efforts to use clinical imaging approaches to predict response to therapy have been limited in their success because the interpretation of imaging is typically qualitative and only based on two time points and/or do not predict oncogene addiction. The objective of the present invention was to develop methods and systems to better predict the efficacy of cancer treatments.

### SUMMARY OF THE INVENTION

The present invention provides technology to predict a tumor's response to a therapy. In clinical practice with this technology, doctors could make earlier determinations of how well a tumor is responding to therapy (e.g. oncogene targeted therapies for hematopoeitic and solid tumors (e.g. lung, kidney or liver). If a patient is not responding well, valuable time could be saved and the patient could be switched to a more efficacious therapy. The techniques of the analysis are objective and quantitative.

Images are acquired of a tumor using an imaging device. Examples of images are CT scans acquired by a CT imaging device, X-rays acquired by an X-ray imaging device, MR images acquired by an MR imaging device, PET scans acquired by a PET imaging device, or ultrasounds acquired by an ultrasound imaging device. The acquisition of these images is performed over time. The timing of the acquisition can be defined in days or weeks. The timing can also be defined as any period of combinations before, during or after the therapy. In one example, embodiments of the invention provide the ability to predict therapeutic outcome within two weeks of the initiation of treatment.

Tumor volumes over the periods of time are determined by analyzing the acquired images over these time periods. Survival signals over time are obtained by analyzing aspects of the tumor over the same time periods. In addition, death signals over time are obtained by analyzing aspects of the tumor over the same time periods. Survival signals and death signals can be obtained using microscopy, immunohistochemistry or imaging.

Cellular proliferation over time is determined by calculating a balance over these time periods of the survival signals and the death signals. Cellular apoptosis over time is determined by calculating a balance over these time periods of the survival signals and said the signals. In one example, cellular proliferation over time and cellular apoptosis over time can each be stochastically determined by the balance over time of the survival signals and the death signals.

A prediction of the tumor's response to therapy is calculated from a combination of (i) the tumor volumes over time, (ii) the cellular proliferation over time and (iii) the cellular apoptosis over time.

### BRIEF DESCRIPTION OF THE DRAWINGS

FIG. 1 shows an overview according to an exemplary embodiment of the invention.

FIG. 2 shows according to an exemplary embodiment of the invention a distribution between three cellular decisions directed by the balance of aggregate survival [S(t)] and death signals [D(t)]. The percentage of cells in each of these states [death (D), homeostasis (H), and proliferation (P)] is determined by thresholds, n and m, and stochastic variability represented by N_{0,1} (standard normal distribution).

FIG. 3 shows according to an exemplary embodiment of the invention distribution changes over time with its mean (line at crest of 3-D mesh) centered at S(t)-D(t), shifting the percentage of cells in the three states.

FIGS. 4A-D show according to an exemplary embodiment of the invention the results generated using normal distribution and log-normal distribution are almost identical. Comparison of the use of normal distribution versus log-normal distribution reveals that both functions produce very similar model fit curves for aggregate survival and death signals as shown in FIG. 2. Regardless of which functions we use to model the behavior of single tumor cells, survival and death signals have similar shapes and decay rates. The model fit well to both of the original tumor volumes using (FIG. 4A) log-normal distribution and (FIG. 4B) normal distribution. The survival signals were short-lived following oncogene inactivation compared to the death signals in both (FIG. 4C) log-normal distribution and (FIG. 4D) normal distribution.

FIG. 5 shows according to an exemplary embodiment of the invention logistic functions, S(t) and D(t), and optimized parameters showed that survival signals were short-lived after oncogene inactivation compared to the death signals.

FIGS. 6A-E show according to an exemplary embodiment of the invention modeling imaging data from the regression of human lung tumors treated with targeted therapy can be used to classify genotype. (FIG. 6A) Quantitative imaging data after simulated oncogene therapy for K-ras^{G12D}—and non-K-ras^{G12D}—induced lung tumors are used to train an SVM algorithm. (FIG. 6B) An illustration of SVM mapping the original data set in a higher-dimensional space, where a maximal separating hyperplane is constructed that best separates the data points between two different genotypes, K-ras^{G12D} and non-K-ras^{G12D}, for classification. (FIG. 6C) Receiver operating characteristic curves show the accuracy of the SVM technique in predicting the oncogene-dependent genotypes based on tumor volumes obtained from different lengths of time after oncogene-targeted therapy. (FIG. 6D) K-ras^{G12D} and non-K-ras^{G12D} tumor volumes over time in orange (top curve) and green (bottom curve), respectively, scaled for differences between mouse and human tumor doubling times (oncogene inactivation at day 0). The black (with top curve) and cyan (with bottom curve) box plots with error bars represent tumor responses from patients with EGFR mutations and wild-type EGFR measured 4 weeks after targeted therapy with erlotinib. Mouse K-ras^{Gl2D} and non-K-ras^{G12D} tumors behave similarly to human tumors with EGFR mutations and wild-type EGFR, respectively, after targeted therapy. This model had an 80% (12 of 15) sensitivity and 100% (28 of 28) specificity for assigning EGFR mutation status. (FIG. 6E) Kaplan-Meier plots of lung cancer patients based on quantitative imaging response at 4 weeks predicted improved PFS (P=0.046).

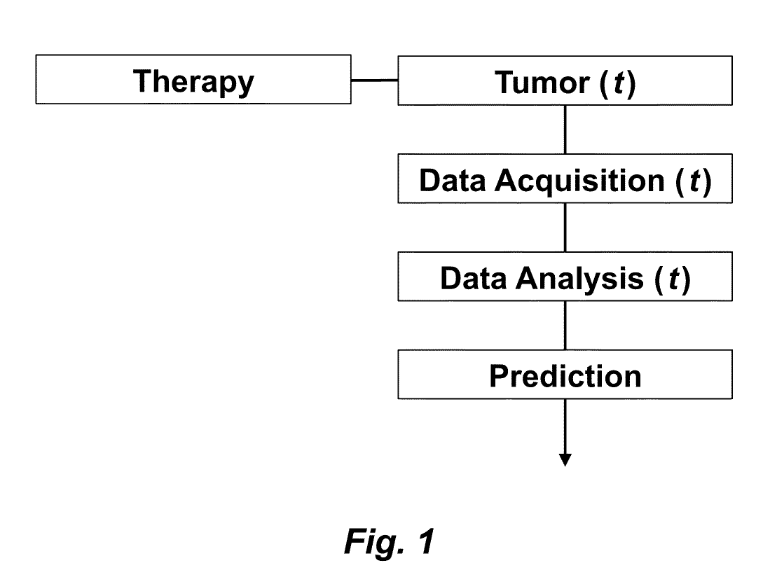

### DETAILED DESCRIPTION

### Model of Signal Behavior

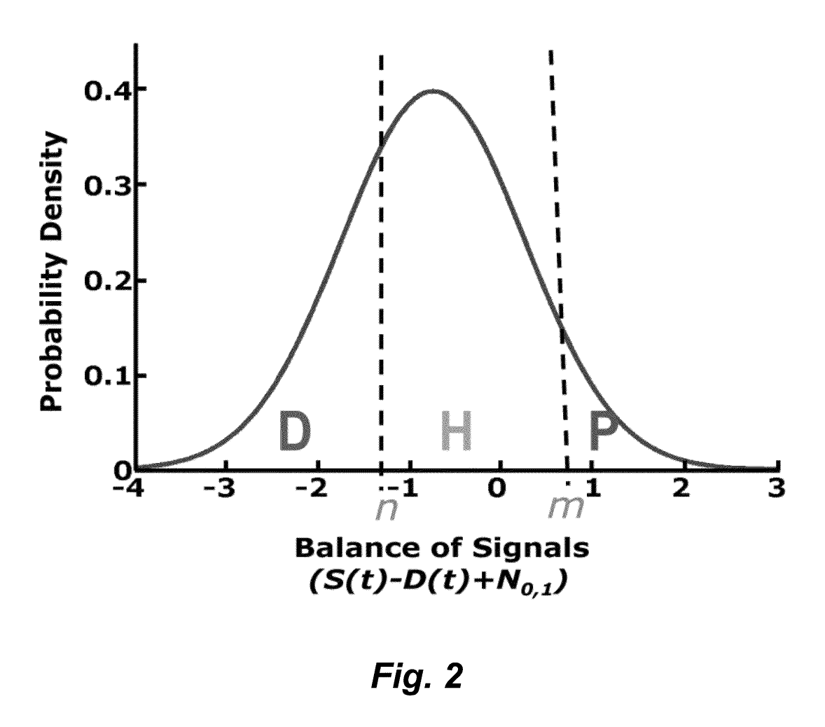

The model of signal behavior represents temporal changes in tumor volumes before and after oncogene inactivation as a balance of two aggregate signals, a survival (S(t)) and a death (D(t)) signal. At any given time cells may react to the balance of these signals through one of three states, proliferation (P), homeostasis (H) or apoptosis (A). The homeostatic population of cells is defined as non-cycling cells and thus may contain cells in G0, the resting phase of the cell cycle, differentiated cells or dormant tumor stem cells (FIG. 2). G0, the proliferation of a cell is organized into several steps; G0 are cells that are resting. G1 are cells that are committed to proliferating. S is when cells make there DNA, G2 is when the cells have doubled their DNA and are about to divide and M is mitosis or cellular divisions

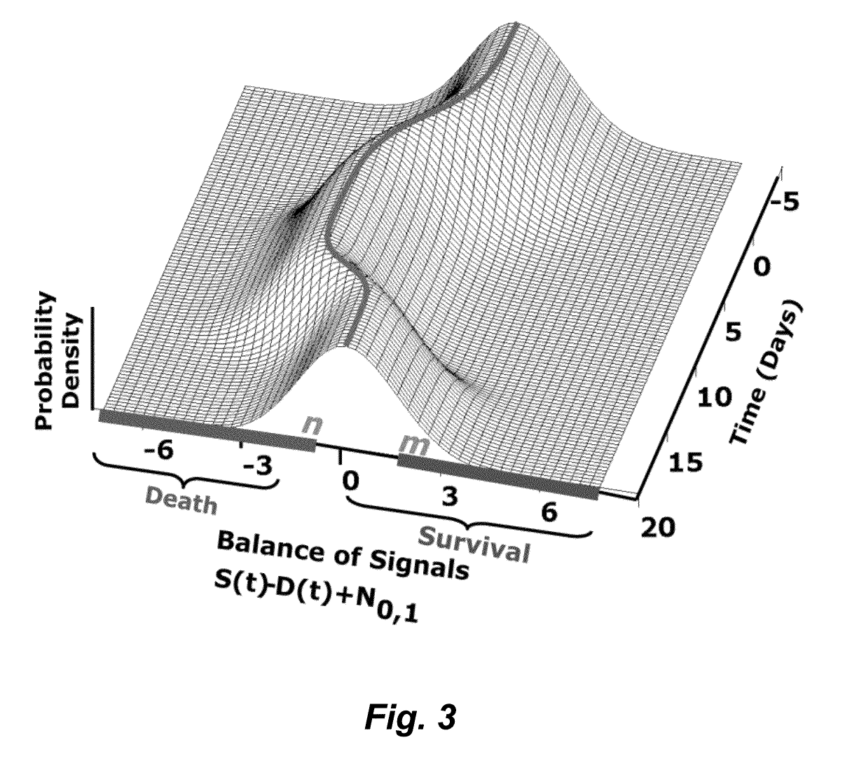

Cells within the same tumor undergo different programmatic decisions such as apoptosis, proliferation, arrest, senescence, differentiation, etc. as is observed empirically with simultaneous proliferation and apoptosis within a given tumor. The stochastic difference in cell behavior to input signals at the microenvironmental level is modeled using a normal distribution CDF Φ(μ,σ²) on the difference between survival and death signals, where μ is the difference between the input signals, and σ is 1. To minimize the number of unknowns and to eliminate a redundant degree of freedom, we fixed the value σ to 1 and thus, the scale of the signaling intensities is in arbitrary units. Along with the normal distribution sampling over time (FIG. 3), the percentage of cells in one of the three states is also determined by two different thresholds, m and n (FIG. 3). We assumed that the large numbers of cells in the tumor are independent random variables. Based on the central limit theorem, the re-averaged sum of the large number of random variables will be approximately distributed normally with finite mean and variance. Therefore, we assumed normal (or Gaussian) distribution to represent the stochastic difference in cell behaviors. In addition, the primary determinants of the fate of cells in this model are the thresholds m and n. Therefore, the specific form of distributions used in the mathematical model should not greatly change the overall prediction.

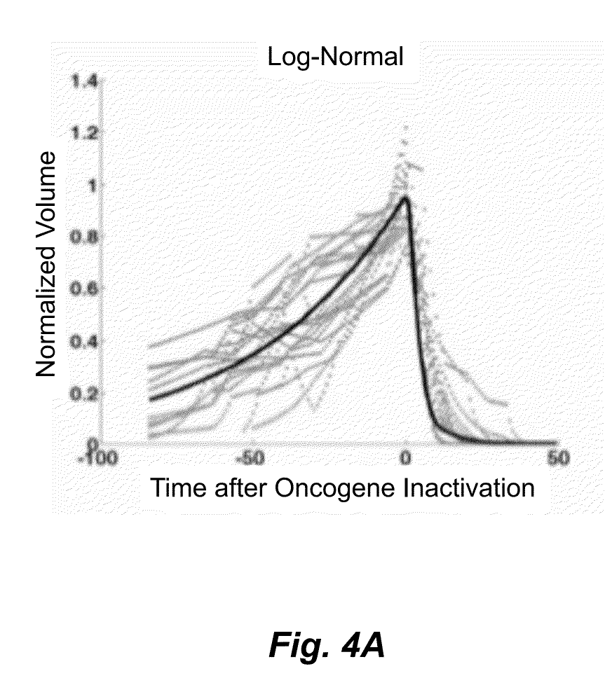

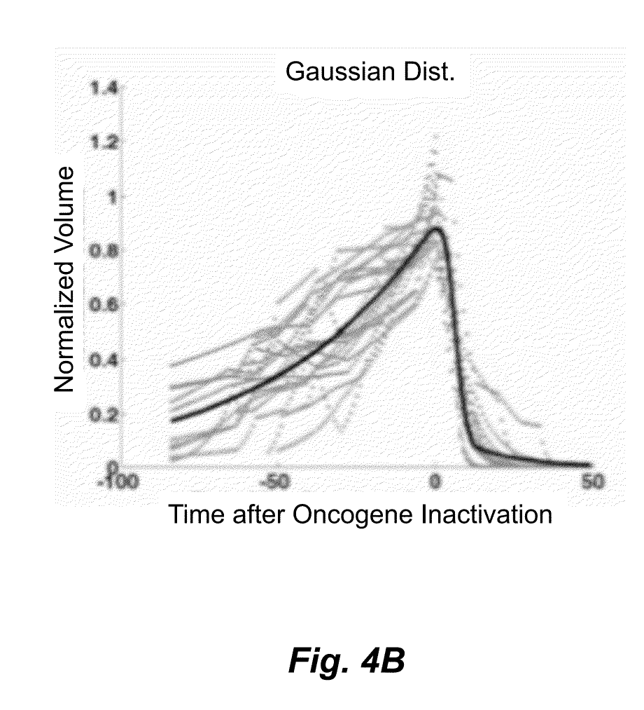

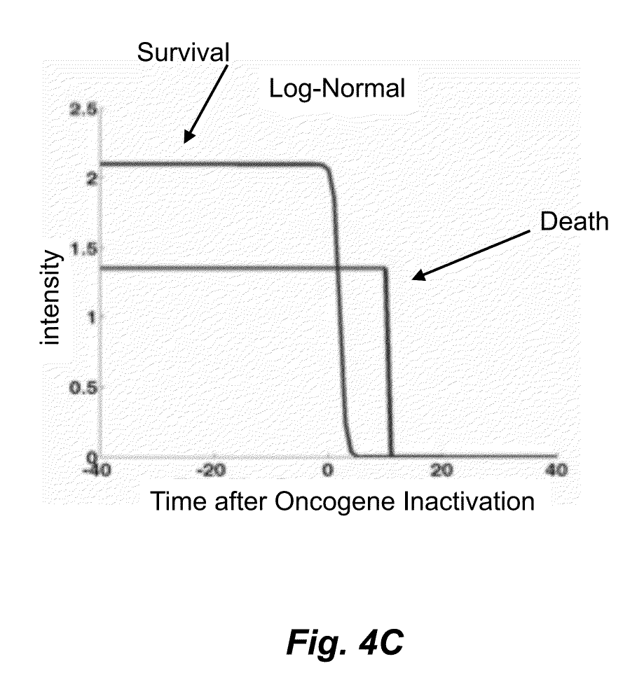

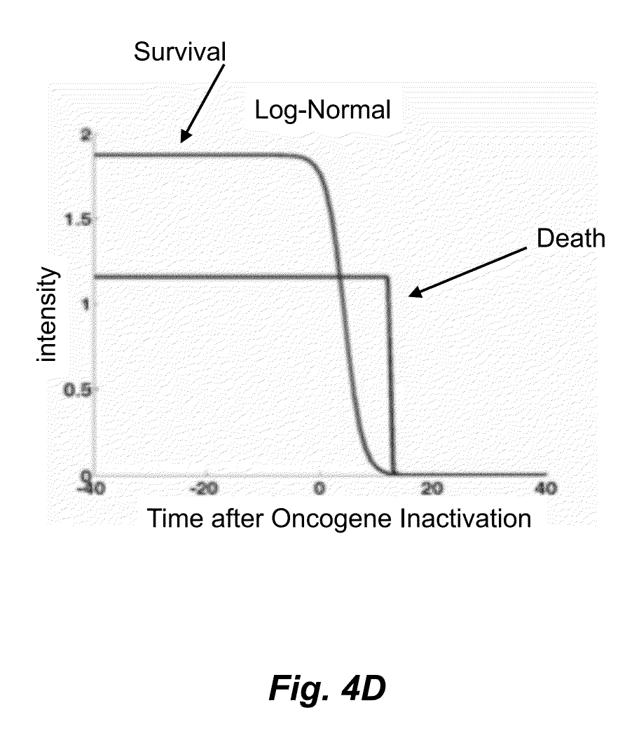

Exemplary comparison of the use of normal distribution versus log-normal distribution reveals that both functions produce very similar model fit curves for aggregate survival and death signals (FIGS. 4A-D). Regardless of which functions we use to model the behavior of single tumor cells, survival and death signals have similar shapes and decay rates. The model fit well to both of the original tumor volumes using (FIG. 4A) log-normal distribution and (FIG. 4B) normal distribution. The survival signals were short-lived following oncogene inactivation compared to the death signals in both (FIG. 4C) log-normal distribution and (FIG. 4D) normal distribution. FIGS. 4A-D show that the results generated using normal distribution and log-normal distribution are almost identical. We used a normal distribution in this examplery embodiment of the invention and for setting up the mathematical model.

The fraction of cells in each state in response to the balance of the input signals can be summarized as follows:

fraction of cells proliferating=1−Φ(m−S(t)+D(t))

fraction of cells in apoptosis=Φ(n−S(t)+D(t)) (1)

fraction of cells in homeostasis=1−(fraction of cells in proliferation)−(fraction of cells in apoptosis)

The rate of volume change over time observed in e.g. the microCT images is therefore determined by the rates of cell proliferation and cell apoptosis, which are then determined by the balance of input signals. These rates can be determined by dividing the number of cells in each state by the amounts of time required for cell proliferation (Tᵥ) and cell apoptosis (Tₐ). Therefore, our model to explain the temporal changes over time based on the balance of the S(t) and D(t) signals can be mathematically summarized by an ordinary differential equation:

$$\frac{d V}{d t} = \frac{1}{{T}_{p}} \cdot ( 1 - Φ ( m - S ( t ) + D ( t ) ) ) \cdot V - \frac{1}{{T}_{a}} \cdot ( Φ ( n - S ( t ) + D ( t ) ) ) \cdot V \qquad ( 2 )$$

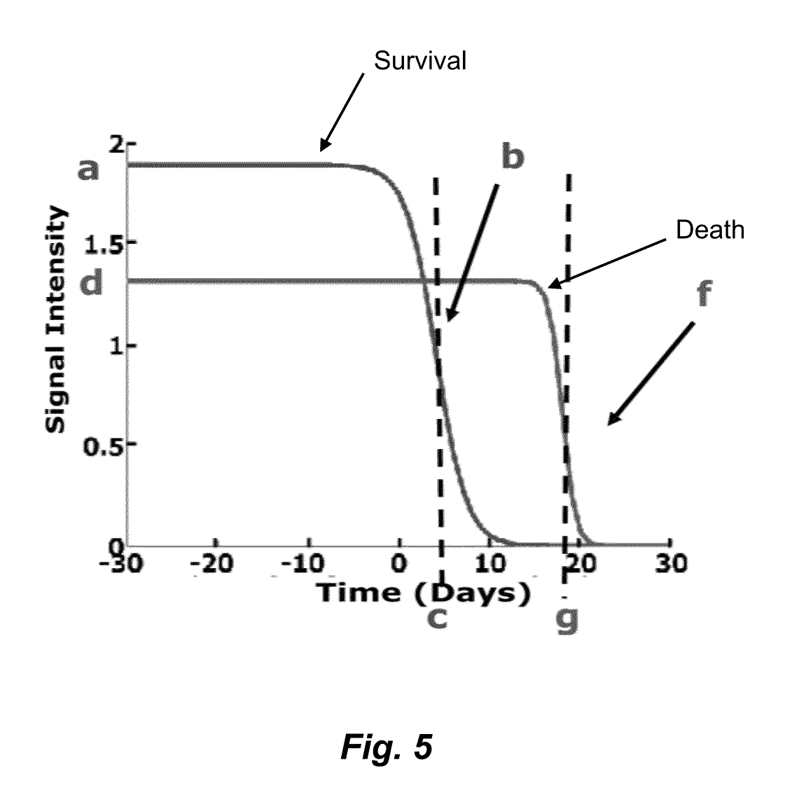

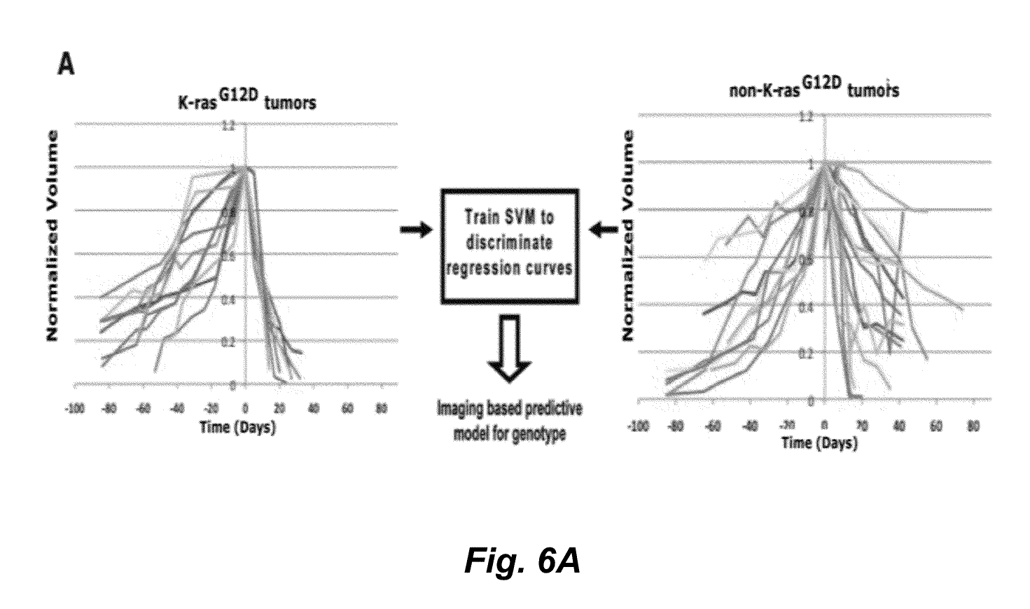

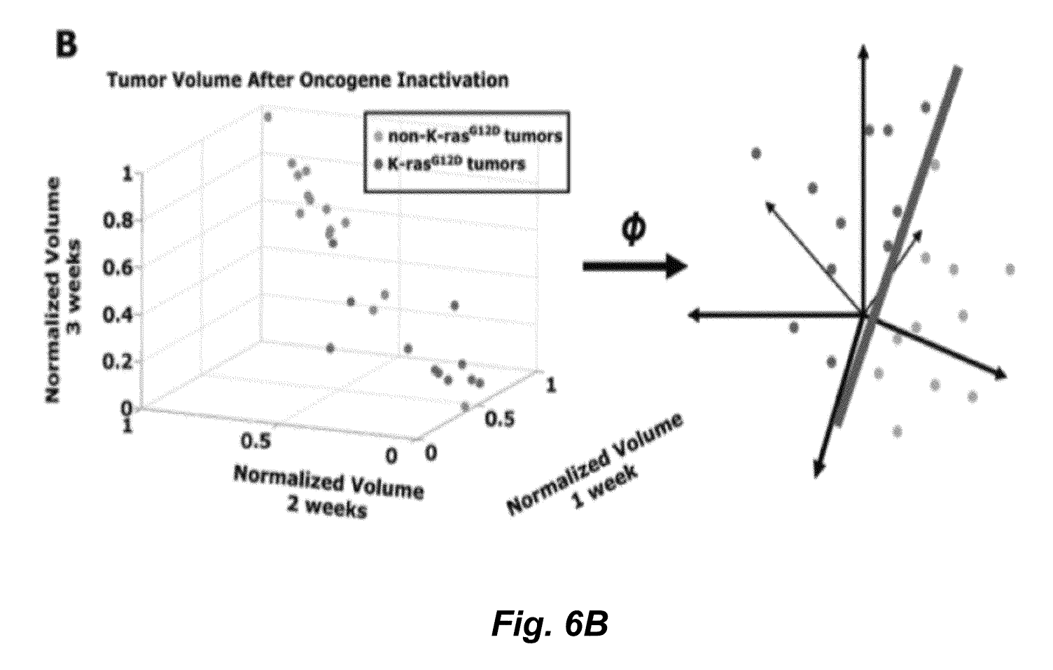

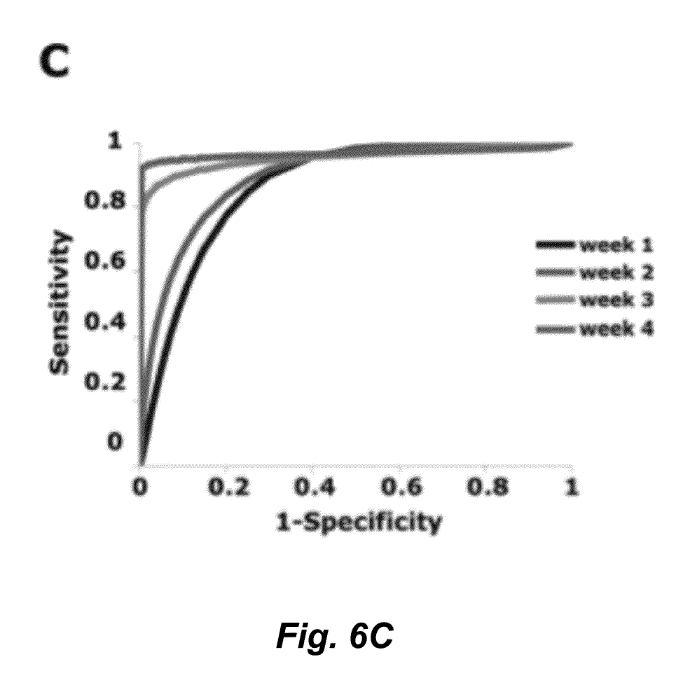

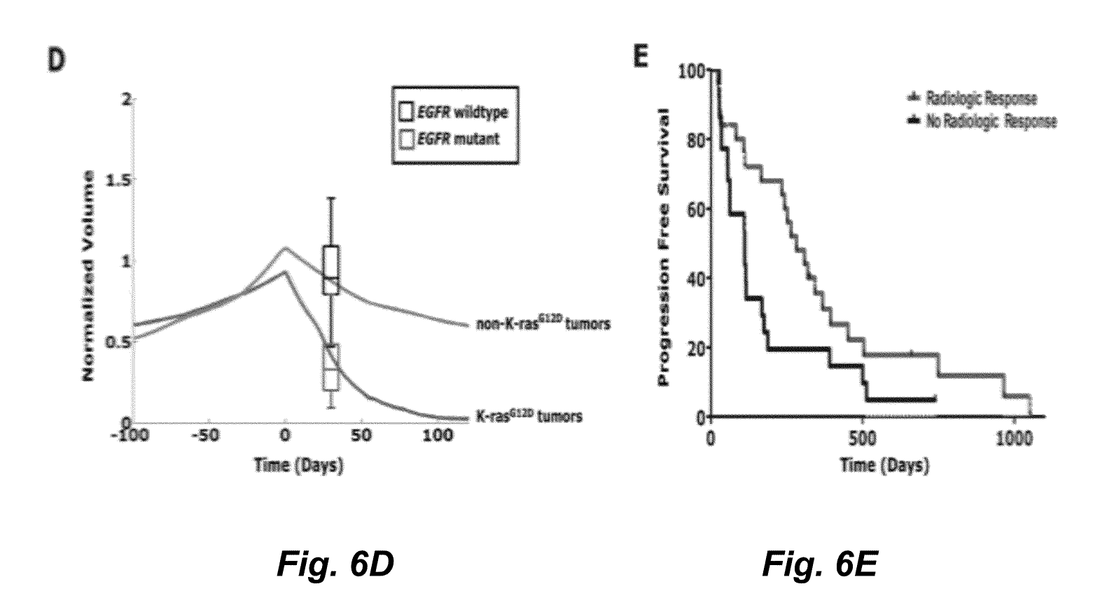

The exponential-like individual tumor volume curves were linearly interpolated in semi-log space to minimize interpolation error. Tumors that were too small to be identified on microCT were treated as a single voxel to avoid log(0). In addition, we modeled the behaviors of the survival S(t) and death D(t) signals as sigmoidal curves because the signals were found to be in a steady state before perturbing the system (inactivation of the oncogene) and we assumed they would reach another steady state some time after perturbation. Therefore, we used a sigmoid function, i.e. the logistic function, which can be mathematically summarized as follows (FIG. 5):

$$S ( t ) = \frac{a}{1 + {e}^{b ( t - c )}} , D ( t ) = \frac{d}{1 + {e}^{f ( t - g )}} \qquad ( 3 )$$

Parameters b and f were the rates of signal decay, c and g were the amounts of time it takes for the signals to begin dropping off, and a and d were the starting intensities of the signals. These parameters were estimated using the Levenberg-Marquardt optimization technique to obtain optimal values based on the actual volumetric measurement obtained from e.g. microCT images and immunohistochemistry data.

Equations (4) and (5) summarizes the temporal rate changes in the immunohistochemistry data, in particular the measurements of cell apoptosis (represented by cleaved caspase 3 and TUNEL staining) and proliferation (represented by Ki-67) (see U.S. Provisional Patent Application 61/629,428 filed Nov. 18, 2011 and herein incorporated by reference for further details). These measurements provide a measure of cells in a state of proliferation and apoptosis, quantified as an instantaneous percentage. However, to quantify the rate as events per unit time, one must also consider the duration for which cells express the markers of proliferation and apoptosis, in this case, tₚ for the duration for which casp-3 is expressed and tₐ or the duration for which Ki-67 is expressed. For example, a fast rate of events with a shorter duration of detectability could have the same instantaneous percentage as a slower rate of events with a longer duration of detectability. Because of a lack of estimates of these durations for these particular tumors in the published literature, we have treated these variables as unknown parameters that are then estimated along with other model parameters. Based on the same assumption for the signaling model, we reasoned that temporal changes in the proliferation and apoptosis rates were also determined by the differences between the survival S(t) and death D(t) signals. Therefore, the relationship between the signals and the IHC measurements can be mathematically summarized by the following equations:

$$\begin{matrix}PI = ( fraction of cells in proliferation ) \cdot \frac{{t}_{p}}{{T}_{p}} = ( 1 - Φ ( m - S ( t ) + D ( t ) ) ) \cdot \frac{{t}_{p}}{{T}_{p} } & ( 4 ) \\ AI = ( fraction of cells in apoptosis ) \cdot \frac{{t}_{a}}{{T}_{a}} = ( Φ ( n - S ( t ) + D ( t ) ) ) \cdot \frac{{t}_{a}}{{T}_{a}} & ( 5 )\end{matrix}$$

where PI is the proliferation index from the exemplary Ki-67 IHC data, AI is the apoptosis index from exemplary cleaved caspase 3 and TUNEL staining IHC data, tₚ and tₐ were the durations that proliferation and apoptosis, respectively, could be detected by immunohistochemistry. In this example, a couple of assumptions were made in the modeling: (1) PI and AI were at a stable rate before oncogene inactivation, and (2) the asymptotic behavior of PI continued beyond 10 days of oncogene inactivation for K-ras^{G12D}-induced lung tumors.

Parameters in equations (2), (4), and (5) were estimated using the Levenberg-Marquardt (LM) algorithm on combined data of volumetric measurements of microCT images and the Ki-67 and cleaved caspase 3 measurements. The task of the LM algorithm can be stated as follows:

Given three sets of data points, Vᵢ (normalized volume), Pᵢ (PI) and Aᵢ (AI), determine all parameters β={a,b,c,d,f,g tₐ, tₚ, Tₐ, Tₚ, n, m} of the model curve f(x,β) to minimize the error function E(β) in equation 6. In other words, parameter values are derived using numerical optimization to match the model output to the experimental output of tumor volume over time, and IHC signals over time.

\hat{V}ᵢ(β), \hat{P}ᵢ(β) and Âᵢ(β) are the fitted values of normalized volume, PI and AI for a given set of parameter values β. To ensure equal weighting between volume, PI and AI datasets, all three were normalized to their maximum value and mean values were used instead of sums.

$$E ( β ) = \sqrt{\frac{1}{{n}_{v}} {\sum }_{i = 1}^{{n}_{v}} {( {\hat{V}}_{i} ( β ) - {V}_{i} )}^{2}} + \frac{1}{max {P}_{i}} \sqrt{\frac{1}{{n}_{p}} {\sum }_{i = 1}^{{n}_{p}} {( {\hat{P}}_{i} ( β ) - {P}_{i} )}^{2}} + \frac{1}{max {A}_{i}} \sqrt{\frac{1}{{n}_{a}} {\sum }_{i = 1}^{{n}_{a}} {( {\hat{A}}_{i} ( β ) - {A}_{i} )}^{2}} \qquad ( 6 )$$

Imaging is performed to determine the overall tumor burden. This can be done, for instance, using CT imaging to measure tumor volume by applying automated segmentation algorithms such as level set methods.

In one example, the death signal is measured using IHC to measure the caspase-3 marker of apoptosis and the survival signal is measured using IHC to measure the Ki-67 marker of proliferation. These markers could also be measured in vivo using molecular imaging probes specific for these markers.

Embodiments of the invention can also be used to examine to predict oncogene addiction PET based imaging that can measure apoptosis and proliferation.

In an additional embodiment, machine learning could be employed using e.g. support vector machines (SVM) to predict tumor responses. For classification, an SVM algorithm with Gaussian kernel was employed as a machine learning classifier.

The steps or algorithms of the embodiments of the invention can be executed on or used with a computer system as a computer-implemented method or as computer-implemented method steps/modules. The computer can receive inputs that are used by the method and/or the computer can provide various outputs, displays or graphics pertaining to the results of the method. Different embodiments can also be manifested as systems combining the computer device and imaging and/or analysis devices.

Additional details or example can be found in the Appendix in U.S. Provisional Patent Application 61/629,428 filed Nov. 18, 2011, which is incorporated herein by reference in its entirety. This Appendix is also published as a paper by the inventors (Tran et al. 2011 entitled “Survival and Death Signals Can Predict Tumor Response to Therapy After Oncogene Inactivation” and published in Sci. Tranl. Med. 3(103) p. 103ra99 (http://m.stm.sciencemag.org/content/3/103/103ra99.abstract). This paper and its full accompanied supplement are incorporated by reference to this application in its entirety.

What is claimed is:

1. A prediction method of a tumor's response to a therapy, comprising:
(a) acquiring images of a tumor using an imaging device, wherein said acquisition of said images is performed over time;
(b) determining tumor volumes using said acquired images, wherein said tumor volumes are determined over said time;
(c) obtaining survival signals over said time by analyzing aspects of said tumor over said time;
(d) obtaining death signals over said time by analyzing aspects of said tumor over said time;
(e) determining cellular proliferation over said time, wherein said cellular proliferation is calculated by a balance over said time of said survival signals and said death signals;
(f) determining cellular apoptosis over said time, wherein said cellular apoptosis is calculated by said balance over said time of said survival signals and said death signals; and
(g) calculating a prediction of said tumor's response to said therapy from a combination of (i) said tumor volumes over said time, (ii) said cellular proliferation over said time and (iii) said cellular apoptosis over said time,
wherein said determining and calculating are computer-implemented method steps performed by a computer.

2. The method as set forth in claim 1, wherein said images are CT scans acquired by a CT imaging device, X-rays acquired by an X-ray imaging device, MR images acquired by an MR imaging device, PET scans acquired by a PET imaging device, or ultrasounds acquired by an ultrasound imaging device.

3. The method as set forth in claim 1, wherein said survival signals and said death signals are obtained using microscopy, immunohistochemistry or imaging.

4. The method as set forth in claim 1, wherein said cellular proliferation over said time and said cellular apoptosis over said time are each stochastically determined by said balance over said time of said survival signals and said death signals.

5. The method as set forth in claim 1, wherein said performed over said time is defined in days or weeks, and wherein said performed over said time is defined as any period of combinations before, during or after said therapy.

6. A system for predicting a tumor's response to a therapy, comprising:
(a) an imaging device for acquiring images of a tumor, wherein said acquisition of said images is performed over time;
(b) a computer device for determining tumor volumes using said acquired images, wherein said tumor volumes are determined over said time;
(c) a data acquisition device for obtaining: (i) survival signals over said time by analyzing aspects of said tumor over said time and (ii) death signals over said time by analyzing aspects of said tumor over said time;
(d) said computer device for determining: (j) cellular proliferation over said time, wherein said cellular proliferation is calculated by a balance over said time of said survival signals and said death signals, and (jj) cellular apoptosis over said time, wherein said cellular apoptosis is calculated by said balance over said time of said survival signals and said death signals; and
(e) said computer device for calculating a prediction of said tumor's response to said therapy from a combination of (k) said tumor volumes over said time, (kk) said cellular proliferation over said time and (kkk) said cellular apoptosis over said time.

7. The system as set forth in claim 6, wherein said images are CT scans acquired by a CT imaging device, X-rays acquired by an X-ray imaging device, MR images acquired by an MR imaging device, PET scans acquired by a PET imaging device, or ultrasounds acquired by an ultrasound imaging device.

8. The system as set forth in claim 6, wherein said data acquisition device is a microscope, an immunohistochemistry analysis device or an imaging device.

9. The system as set forth in claim 6, wherein said cellular proliferation over said time and said cellular apoptosis over said time are each stochastically determined by said balance over said time of said survival signals and said death signals.

10. The system as set forth in claim 6, wherein said performed over said time is defined in days or weeks, and wherein said performed over said time is defined as any period of combinations before, during or after said therapy.
